(19) United States
(12) Patent Application Publication
(10) Pub. No.: US 2007/0178470 A1
(43) Pub. Date: Aug. 2, 2007

(54) System for charge-based detection of nucleic acids

(75) Inventors: Luc Bissonnette, Quebec (CA); Frederic Raymond, Montpellier (FR); Regis Peytavi, St-Romuald (CA)

Correspondence Address: GODFREY & KAHN S.C., 780 NORTH WATER STREET, MILWAUKEE, WI, 53202, US

(73) Assignee: INFECTIO RECHERCHE INC., Ste-Foy, QC (CA)

(21) Appl. No.: 10/596,397

(22) PCT Filed: Dec. 13, 2004
(86) PCT No.: PCT/CA04/02118

§ 371 Date: Apr. 19, 2007

Related U.S. Application Data
(60) Provisional application No. 60/528,748, filed on Dec. 12, 2003.

(51) Int. Cl. C12Q 1/68; C12M 1/34

(52) U.S. Cl. 435/6; 435/287.2

(57) ABSTRACT

This present invention relates methods for detecting the presence of nucleic acids in a sample. In these methods, neutral capture probes are exposed to a sample possibly containing complementary nucleic acid targets. The foregoing mixture is submitted to conditions that provide for the nucleic acid targets to bind with the neutral probes thereby generating hybrids. These hybrids are submitted to positively charged reporters such as atoms, molecules or macromolecules, which electrostatically bind to the hybrids. The complexes formed between reporters and hybrids are detected by a variety of detection methods. Kits for detecting the presence of nucleic acids in a sample are also disclosed herein.

### FIELD OF THE INVENTION

The present invention relates to a system for charge-based detection of nucleic acids.

### BACKGROUND OF THE INVENTION

1. Methods for Detection of Nucleic Acids

The recombinant DNA technology era has provided researchers and biotechnology-oriented industries several important methods for the specific detection of nucleic acids. Molecular hybridization methods, nucleic acid amplification technologies, and more recently, microarray and biochip technologies are known to those skilled in the art.

Examples of molecular hybridization techniques include the Southern and Northern blotting methods in which electrophoretically separated DNA or RNA macromolecules are generally transferred from a gel matrix and fixed to a membrane filter made of nitrocellulose or nylon, and made available for hybridization with radiolabeled, fluorescent, or biotinylated nucleic acid probes, potentially complementary to transferred molecular species (Sambrook and Russel, 2001, Molecular Cloning: A laboratory manual (Third edition), Cold Spring Harbor Laboratory Press, New York, N.Y., pp. 6.39-6.50, pp. 7.42-7.45).

Examples of nucleic acid amplification technologies include the polymerase chain reaction (PCR) and derived methods (reverse transcriptase-PCR, real-time PCR), NASBA, SDA, etc., methods which permit to selectively amplify parts of a nucleic acid molecule between oligodeoxyribonucleotide primers, and in some instances, allow for concomitant detection (Nolte and Caliendo, 2003, Molecular detection and identification of microorganisms, pp. 234-256, In Manual of Clinical Microbiology (8^{th} ed.), Murray et al., American Society for Microbiology, Washington, D.C.; Fredricks and Relman, 1999, Clin. Infect. Dis., 29:475-488).

More recently, robotic spotters, electric field control, and photolithographic methods have been used to spot, direct, or chemically-synthesize deoxyribonucleotide probes at the surface of various solid supports or devices. Such modified supports (glass or silicon slides) or devices (Nanogen electrically active microchips, Affymetrix biochips, etc.) are then subjected to hybridization with samples containing sought amplified genetic targets and treated to reveal hybridization signals (Jain, 2000, Pharmacogenomics, 1:289-307; Vo-Dinh and Collum, 2000, Fresenius J. Anal. Chem., 366:540-551).

Overall, these methods have significantly contributed to advances in molecular biology, but for diagnostic applications, their use is hampered by either lack of speed, sensitivity, or practicality.

Microarray and biochip technologies offer great potential for multi-parametric detection since up to several thousands of capture probes can be immobilized or synthesized at the surface of a solid support such as glass or silicon. These probes can then serve as complementary ligands for hybridization to amplified (and generally labeled) nucleic acids from the sample.

A simpler strategy for nucleic acids detection on microarray would reside in a system where nucleic acids from sought-after genetic targets, once hybridized to capture probes, would provide a scaffold for the electrostatic recognition of the negatively-charged phosphates by binding of atoms, molecules, or macromolecules, and the formation and subsequent detection of higher order complexes by optical, fluorescent, or electrochemical methods or devices. However, on a solid support, the use of capture probes made of deoxyribonucleotides (dNTPs) would result in a background signal due to the presence of negatively-charged phosphate groups that would react with the reporter atoms, molecules, or macromolecules.

Kinetically speaking, the use of uncharged probes contributes to increase the rate of hybridization of the nucleic acids from the samples by alleviating the repulsion of negatively-charges nucleic acid strands in classical hybridization (Nielsen et al., 1999, Curr. Issues Mol. Biol., 1:89-104). The generation of easily detectable higher-order complexes along the scaffold of hybridized nucleic acids from the sought after genetic targets serves to increase the relative mass of the capture probe-nucleic acid target, and hence, the sensitivity of the system (Sastry, 2002, Pure Appl. Chem., 74:1621-1636 Xiao et al., 2002, J. Nanoparticle Res., 4:313-317).

2. Uncharged Deoxyribonucleotide Analogs

2.1 Peptide Nucleic Acids (PNA)

PNAs are nucleic acid analogs for which the phosphodiester backbone has been replaced by a polyamide, which makes PNAs a polymer of 2-aminoethyl-glycine units bound together by an amide linkage. PNAs are synthesized using the same Boc or Fmoc chemistry as are use in standard peptide synthesis. Bases (adenine, guanine, cytosine and thymine) are linked to the backbone by a methylene carboxyl linkage. Thus, PNAs are acyclic, achiral, and neutral. Other properties of PNAs are increased specificity and melting temperature as compared to nucleic acids, capacity to form triple helices, stability at acid pH, non-recognition by cellular enzymes like nucleases, polymerases, etc. (Rey et al., 2000, FASEB J., 14:1041-1060; Nielsen et al., 1999, Curr. Issues Mol. Biol., 1:89-104). The possibility of building PNA microarrays, for detection of unlabeled and labelled nucleic acid samples, was investigated by several researchers, as recently reviewed by Brandt and Hoheisel (Brandt and Hoheisel, 2004, Trends Biotechnol, 22:617-622). However, detection of hybridization was achieved by using labeled analytes (Brandt et al., 2003, Nucl. Acids Res., 31:e109; Germini et al., 2004, J Agric Food Chem, 52:4535-4540) and although detection of solid support bound PNA hybridized to unlabeled DNA could be achieved, it required complex technologies, such as time-of-flight secondary ion mass spectrometry (TOF-SIMS; Brandt at al., 2003, Nucl. Acids Res., 31:e119) or quartz crystal microbalance (QCM; Wang et al., Anal. Chem., 1997, 69:5200-5202).

2.2 Methylphosphonate Nucleotides

Methylphosphonates are neutral DNA analogs containing a methyl group in place of one of the non-bonding phosphoryl oxygens. Oligonucleotides with methylphosphonate linkages were among the first reported to inhibit protein synthesis via anti-sense blockade of translation. However, the synthetic process yields chiral molecules that must be separated to yield chirally pure monomers for custom production of oligonucleotides (Reynolds et al., 1996, Nucleic Acids Res., 24:4584-4591).

3.0 Reporter Atoms and Molecules

Multiparametric nucleic acid detection using microarray platforms are currently mostly being performed using commercially available fluorescence readers. However, classical strategies require labeling the analyte or the probes with fluorophores or other reporting molecules. This labeling approach renders the reaction mixture more complex, and reduces sensitivity and specificity (Brandt and Hoheisel, 2004, Trends Biotechnol., 22:617-622).

4. Nucleic Acid Detection Methods Relying on Molecular Charge

Deoxyribonucleic acid (DNA) and ribonucleic acid (RNA) are polymers of nucleotides which are composed of a phosphodiester backbone to which bases are linked (adenine, guanine, cytosine, and thymine). The phosphate moieties of the backbone are responsible for the negative charge of DNA and RNA (Voet and Voet. 1995. Biochemistry (Second Edition), John Wiley and Sons Inc, New York, N.Y.). Methods have been used to detect unlabeled DNA by virtue of it's anionic nature. Examples of these methods are described below.

4.1 Electronic Detection

Electronic detection of DNA using microfabricated silicon field-effect sensors to monitor the increase in surface charge when a DNA oligomer hybridizes to a complementary oligodeoxyribonucleotide bound to a sensor surface have been developed (Fritz et al., 2002, Proc. Natl. Acad. Sci. U.S.A., 99:14142-14146).

4.2 Time-of-flight Secondary Ion Mass Spectrometry

Detection of unlabeled DNA hybridized to PNA probes using mass spectrometry has been reported (Brandt et al., 2003, Nucleic Acids Res., 31:e119). Glass bound PNA oligomers are hybridized to complementary oligonucleotides. Using time-of-flight secondary ion mass spectrometry (TOF-SIMS) to detect DNA's phosphates, PNA-DNA and PNA-RNA duplexes can be discriminated from unhybridized PNA.

4.3 Conjugated Polymers

Novel approaches were developed for DNA/RNA detection based on electrostatic interactions between cationic polymers and nucleic acids (Pending patent application PCT/CA02/00485; Ho et. al., 2002, Angew. Chem. Int. Ed., 41:1548-1551; Ho et al., 2002, Polymer Preprints, 43:133-134). These new approaches exploit a modification of the optical or electrochemical properties of polymer biosensors upon electrostatic binding to a single- or a double-stranded negatively-charged nucleic acid molecule. These macromolecular interactions are associated with conformational and solubility changes which contribute to signal generation (Ho et. al., 2002, Angew. Chem. Int. Ed., 41:1548-1551). These polymer-based detection technologies do not require any chemical labeling of the probe or of the target and can discriminate between specific and non-specific hybridization of nucleic acids that differ by a single nucleotide acid (Pending patent application PCT/CA02/00485; Ho et. al., 2002, Angew. Chem. Int Ed., 41:1548-1551; Ho et al., 2002, Polymer Preprints, 43:133-134).

A similar method using water-soluble fluorescent zwitterionic polythiophene derivatives has been reported (Nilsson et al., 2003, Nat. Mater. 2:419-424). This kind of polymer has also been used to detect DNA bound to gel pads. DNA oligomers are electrostatically bound to polythiophene derivatives and then incorporated into gel pads. After hybridization to complementary oligonucleotides, a shift in fluorescence is observed.

Other water soluble cationic conjugated polymers, polyfluorene phenylene (Gaylord et al., 2002, Proc. Natl. Acad. Sci. U.S.A., 99:10954-10957) and poly(3,4-ethylenedioxythiophene) (Krishnamoorthy et al., 2004, Chem. Commun., 2004:820-821), have been used for the detection of unlabeled nucleic acids.

However, detection technologies taking advantage of the anionic properties of nucleic acids suffer from undesirable background noise caused by the capture probes. There thus remains a need to develop a system for the charge-based detection of nucleic acids having reduced background noise.

The present invention seeks to meet these and other needs. It refers to a number of documents, the content of which is herein incorporated by reference in their entirety.

### SUMMARY OF THE INVENTION

The present invention relates to the use of neutral analogs of nucleic acids such as peptide nucleic acid (PNA) or methylphosphonates. These neutral analogs of nucleic acids (such as neutral capture probes), when used in combination with reporters such as cationic polymers (for example electroactive cationic polythiophenes; see FIG. 1A for structure of monomer basic unit) lead to a better signal since the polythiophenes do not bind to the neutral probes and will only recognize the anionic hybridized nucleic acids from the analyte (nucleic acid targets),

The present invention relates to the detection of unlabeled nucleic acids that hybridize to neutral nucleic acid analogs (such as probes that are complementary to the targeted nucleic acids from a sample) bound onto surfaces, such as probe arrays (e.g. microarrays).

The present invention also relates to a method of detecting unlabeled nucleic acids, using reporter atoms, molecules or macromolecules including fluorescent, electroactive, water-soluble, cationic polythiophene derivatives, which electrostatically bind to unlabeled negatively-charged nucleic acids (e.g. DNA, RNA, etc.), hybridized to a neutral nucleic acid analog that is bound to a surface.

Additionally, the present invention relates to a method for detecting hybridization of unlabeled nucleic acids to a neutral nucleic acid analog probe using transducers such as the reporters of the present invention.

Furthermore, the present invention relates to the use of probes made of uncharged deoxyribonucleotide analogs.

Moreover, the present invention relates to a reagent kit for the detection of nucleic acids hybridizing to neutral nucleic acids analog oligomers immobilized onto a solid support.

In accordance with an aspect of the present invention, there is provided a method for detecting the presence of nucleic acids in a sample, this method comprising:
(a) exposing uncomplexed neutral capture probes to a sample possibly containing complementary nucleic acid targets, thereby generating a mixture;
(b) submitting this mixture to hybridization conditions which provide for said nucleic acids targets to bind specifically to complementary neutral capture probes, thereby generating negatively charged capture probe-nucleic acid target hybrids;
(c) submitting these negatively charged hybrids to positively charged reporters selected from group consisting of transition metal atoms, molecules, or macromolecules being capable of electrostatically binding to said hybrids, thereby generating higher-order complexes; and
(d) detecting said higher-order complexes.

In accordance with another aspect of the present invention, there is provided a method for detecting the presence of nucleic acids in a sample, this method comprising:
(a) exposing uncomplexed neutral capture probes to a sample possibly containing complementary nucleic acid targets and containing positively charged reporters selected from group consisting of transition metal atoms, molecules or macromolecules, thereby generating a mixture;
(b) submitting this mixture to hybridization conditions which provide for the nucleic acids targets to bind specifically to complementary neutral capture probes, thereby generating negatively charged capture probe-nucleic acid target hybrids, the reporters being capable of electrostatically binding to the hybrids, thereby generating higher-order complexes; and
(c) detecting these higher-order complexes.

In accordance with a further aspect of the invention, there is provided a kit for detecting the presence of nucleic acids in a sample, this kit comprising:
uncomplexed neutral capture probes;
a control sample possibly containing nucleic acid targets that are complementary to the neutral capture probes; and
one or more positively charged reporters selected from the group consisting of transition metal atoms, molecules or macromolecules; these reporters being capable for electrostatically binding to negatively charged capture probe-nucleic acid target hybrids,

In an embodiment, a washing step is performed after reporters have been exposed to probe-target hybrids.

In an embodiment, the nucleic acids targets are unlabeled. In an embodiment, the nucleic acid targets comprise DNA or RNA molecules In an embodiment, the nucleic acid targets are generated by chemical synthesis or molecular biology methods selected from the group consisting of polymerase chain reaction (PCR), reverse transcriptase-PCR (RT-PCR), strand displacement amplification (SDA), ligase chain reaction (LCR), transcription-associated amplification, nucleic acid sequence-based amplification (NASBA), whole genome amplification (WGA), helicase-dependent isothermal amplification, or other methods known by those skilled in the art.

In an embodiment, the capture probes are immobilized on a support surface. In an embodiment, the neutral capture probes are chemically modified to incorporate a functional group providing for the probes to covalently link to the surface. In an embodiment, the functional group is selected from the group consisting of amine, aldehyde, thiol, epoxy or carboxyl moieties. In an embodiment, the neutral capture probes are selected from the group consisting of peptide nucleic acids (PNA) and methylphosphonate.

In an embodiment, the support surface is selected from the group consisting of a glass surface, a silicon surface, a gold surface, an electrode surface, a particle surface, a gel matrix, a membrane surface, a paper surface or a plastic surface. In an embodiment, the support surface comprises a solid support surface. In an embodiment, the solid support surface comprises a probe array. In an embodiment, the solid support is coated with a passivation agent preventing non-specific binding of nucleic acid targets. In an embodiment, this passivation agent is selected from the group consisting of polyvinylpyrollidone, polyethylene glycol, and BSA. In an embodiment, the solid support surface is chemically modified, to facilitate coupling and chemical bonding of the neutral probe to the solid support surface. In an embodiment, the solid support surface is chemically modified to yield functional groups selected from the group consisting of: an aldehyde, an aminoalkylsilane activated with carbonyldiimidazole, thiol, epoxy or carboxyl moieties.

In an embodiment, PNA are hybridized to amplicon produced using design rules described in the co-pending application (U.S. patent application No. 60/592,392). These rules include more stringent conditions such as: smaller size of the amplicon (<300 bp); amplicon centered or directed toward the slide surface. Additionally, single-stranded analyte nucleic acids can be used to minimize the destabilizing effect of the complementary strand.

In an embodiment, the reporters serve as transducers since cationic polythiophene polymers are known to exhibit differential colorimetric, electrochemical, and fluorescence properties upon binding to nucleic acids. In an embodiment, the reporters exhibit low affinity for uncharged probes. In an embodiment, the reporters are capable of electrostatically binding to the phosphate backbone of the hybrids. In an embodiment, the reporters comprise polythiophenes (see FIG. 1A). In an embodiment, the polythiophenes are water soluble and cationic. In an embodiment, the reporters comprise enzymes. In an embodiment, these enzymes comprise alkaline phosphatase and polystyrene beads conjugated thereto.

In an embodiment, the transition metal cations used as reporters are selected from the group consisting of Ag⁺, Cd⁺⁺, or other ions that can be chemically modified to yield higher-order complexes using bound nucleic acids as a scaffold.

In an embodiment, detection includes a chemical reaction step rendering the transition metal cations detectable. For example, Ag⁺ can De reduced to Ag⁰ and Cd⁺⁺ can react with H₂S or Na₂S to yield CdS quantum dots, in conditions that prevent the dissociation of hybridized nucleic acids or nucleic acids-PNA duplexes.

In an embodiment, the enzymes comprise alkaline phosphatase and polystyrene beads conjugated thereto. In an embodiment, detection is selected from the group consisting of optical detection, fluorometic detection, colorimetric detection, electrochemical detection, chemiluminescent detection, microscopy or spectrophotometric detection.

Further scope and applicability will become apparent from the detailed description given hereinafter. It should be understood however, that this detailed description, while indicating preferred embodiments of the invention, is given by way of illustration only, since various changes and modifications within the spirit and scope of the invention will become apparent to those skilled in the art.

Definitions

Unless defined otherwise, the scientific and technical terms and nomenclature used herein have the same meaning as commonly understood by a person of ordinary skill to which this invention pertains. Commonly understood definitions of molecular biology terms can be found for example in Dictionary of Microbiology and Molecular Biology, 2nd ed. (Singleton et al., 1994, John Wiley & Sons, New York, N.Y.), the Harper Collins Dictionary of Biology, Hale & Marham, 1991, Harper Perennial, New York, N.Y.); Rieger et al., Glossary of genetics: Classical and molecular, 5^{th} edition, Springer-Verlag, New York, 1991;

Alberts et al., Molecular Biology of the Cell, 40^{th} edition, Garland science, New York, 2002; and, Lewin, Genes VII, Oxford University Press, New York, 2000. Generally, the procedures of molecular biology methods and the like are common methods used in the art. Such standard techniques can be found in reference manuals such as for example Sambrook et al. (2000, Molecular Cloning—A Laboratory Manual, Third Edition, Cold Spring Harbor Laboratories); and Ausubel et al. 1994, Current Protocols In Molecular Biology, Wiley, New York).

In the present description, a number of terms are extensively utilized, In order to provide a clear and consistent understanding of the specification and claims, including the scope to be given such terms, the following definitions are provided.

The use of the word “a” or “an” when used in conjunction with the term “comprising” in the claims and/or the specification may mean “one” but it is also consistent with the meaning of “one or more”, “at least one”, and “one or more than one”.

The use of the term “or” in the claims is used to mean “and/or” unless explicitly indicated to refer to alternatives only or the alternatives are mutually exclusive, although the disclosure supports a definition that refers to only alternatives and “and/or”.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

In the claims, unless otherwise specified the plural form includes the singular form and vice versa.

As used herein, “nucleic acid targets”, “nucleic acid molecule” or “polynucleotides”, refers to a polymer of nucleotides. Non-limiting examples thereof include DNA (e.g. genomic DNA, cDNA), RNA molecules (e.g. mRNA) and chimeras thereof. The nucleic acid targets can be obtained from a sample. The nucleic acid targets can be obtained by cloning techniques or synthesized. DNA can be double-stranded or single-stranded (coding strand or non-coding strand [antisense]). Conventional ribonucleic acid (RNA) and deoxyribonucleic acid (DNA) are included in the term “nucleic acid” and polynucleotides as are analogs thereof, A nucleic acid backbone may comprise a variety of linkages known in the art, including one or more of sugar-phosphodiester linkages, peptide-nucleic acid bonds (referred to as “peptide nucleic acids” (PNA); Hydig-Hielsen et al., PCT Int'l Pub. No. WO 95/32305), phosphorothioate linkages, methylphosphonate linkages or combinations thereof. Sugar moieties of the nucleic acid may be ribose or deoxyribose, or similar compounds having known substitutions, e.g. 2′ methoxy substitutions (containing a 2′-O-methylribofuranosyl moiety; see PCT No. WO 98/02582) and/or 2′ halide substitutions. Nitrogenous bases may be conventional bases (A, G, C, T, U), known analogs thereof (e-g., inosine or others; see The Biochemistry of the Nucleic Acids 5-36, Adams et al., ea., 11^{th} ed., 1992), or known derivatives of purine or pyrimidine bases (see, Cook, PCT Int'l Pub. No. WO 93/13121) or “abasic” residues in which the backbone includes no nitrogenous base for one or more residues (Arnold et al., U.S. Pat. No. 5,585,481). A nucleic acid may comprise only conventional sugars, bases and linkages, as found in RNA and DNA, or may include both conventional components and substitutions (e.g., conventional bases linked via a methoxy backbone, or a nucleic acid including conventional bases and one or more base analogs).

As used herein, “oligomers”, “oligonucleotides” or “oligos” define a molecule having two or more nucleotides (ribo or deoxyribonucleotides). The size of the oligo will be dictated by the particular situation and ultimately on the particular use thereof and adapted accordingly by the person of ordinary skill An oligonucleotide can be synthesized chemically or derived by cloning according to well known methods. While they are usually in a single-stranded form, they can be in a double-stranded form and even contain a “regulatory region”. They can contain natural rare or synthetic nucleotides. They can be designed to enhance a chosen criteria like stability for example.

Nucleic acid hybridization. Nucleic acid hybridization depends on the principle that two single-stranded nucleic acid molecules that have complementary base sequences will reform the thermodynamically favored double-stranded structure if they are mixed under the proper conditions. The double-stranded structure will be formed between two complementary single-stranded nucleic acids even if one is immobilized on a nitrocellulose filter. In the Southern or Northern hybridization procedures, the latter situation occurs. The DNA/RNA of the individual to be tested may be digested with a restriction endonuclease, prior to its fractionation by agarose gel electrophoresis, conversion to the single-stranded form, and transfer to nitrocellulose paper, making it available for reannealing to the hybridization probe. Non-limiting examples of hybridization conditions can be found in Ausubel, F. M. et al., Current protocols in Molecular Biology, John Wiley & Sons, Inc., New York, N.Y. (1994). A nitrocellulose filter is incubated overnight at 68° C. with labeled probe in a solution, high salt (either 6×SSC[20×: 3M NaCl/0.3M trisodium citratel or 6×SSPE [2×: 3.6M NaCl/0.2M NaH₂PO₄/0.02M EPTA, pH 7.7]). 5× Denhardt's solution, 0.5% SDS, and 100 μg/mL denatured salmon sperm DNA. This is followed by several washes in 0.2×SSC/0.1% SDS at a temperature selected based on the desired stringency: room temperature (low stringency), 42° C. (moderate stringency) or 68° C. (high stringency). The salt and SPS concentration of the washing solutions may also be adjusted to accommodate for the desired stringency. The temperature and salt concentration selected is determined based on the melting temperature (Tm) of the DNA hybrid. Other protocols or commercially available hybridization kits using different annealing and washing solutions can also be used as well known in the art. “Nucleic acid hybridization” refers generally to the hybridization of two single-stranded nucleic acid molecules having complementary base sequences, which under appropriate conditions will form a thermodynamically favored double-stranded structure. Examples of hybridization conditions can be found in the two laboratory manuals referred above (Sambrook et al., 2000, supra and Ausubel et al., 1994, supra) and are commonly known in the art. In the case of a hybridization to a nitrocellulose filter (or other such support like nylon), as for example in the well known Southern blotting procedure, a nitrocellulose filter can be incubated overnight at 65° C. with a labeled probe in a solution containing high salt (6×SSC or 5×SSPE), 5× Denhardt's solution, 0.5% SDS, and 100 μg/mL denatured carrier DNA (e.g. salmon sperm DNA). The non-specifically binding probe can then be washed off the filter by several washes in 0.2×SSC/0.1% SDS at a temperature which is selected in view of the desired stringency: room temperature (low stringency), 42° C. (moderate stringency) or 65° C. (high stringency), The salt and SDS concentration of the washing solutions may also be adjusted to accommodate for the desired stringency. The selected temperature and salt concentration is based on the melting temperature (Tm) of the DNA hybrid. Of course, RNA-DNA hybrids can also be formed and detected. In such cases, the conditions of hybridization and washing can be adapted according to well known methods by the person of ordinary skill. Stringent conditions will be preferably used (Sambrook et al., 2000, supra). Other protocols or commercially available hybridization kits (e.g., ExpressHyb™ from BD Biosciences Clontech) using different annealing and washing solutions can also be used as well known in the art.

By “complementary” or “complementarity” or “analog” is meant that nucleic acid can form hydrogen bond(s) with another nucleic acid sequence by either traditional Watson-Crick base pairing or other non-traditional types of interactions. In reference to the nucleic acid molecules of the present invention, the binding free energy for a nucleic acid molecule with its complementary sequence is sufficient to allow the relevant function of the nucleic acid to proceed (e.g., RNAi activity). For example, the degree of complementarity between the sense and antisense region (or strand) of the siRNA construct can be the same or can be different from the degree of complementarity between the antisense region of the siRNA and the target RNA sequence (e.g., Staufen RNA sequence). Complementarity to the target sequence of less than 100% in the antisense strand of the siRNA duplex (including deletions, insertions, and point mutations) is reported to be tolerated when these differences are located between the 5′-end and the middle of the antisense siRNA (Elbashir et al., 2001, EMBO J., 20:6877-6888). Determination of binding free energies for nucleic acid molecules is well known in the art (e.g., see Turner et al., 1987, J. Am. Chem. Soc., 190:3783-3785; Frier et al., 1986, Proc. Natl. Acad. Sci. U.S.A., 83 :9373-9377) “Perfectly complementary” means that all the contiguous residues of a nucleic acid molecule will hydrogen bond with the same number of contiguous residues in a second nucleic acid sequence.

By “sufficiently complementary” is meant a contiguous nucleic acid base sequence that is capable of hybridizing to another sequence by hydrogen bonding between a series of complementary bases. Complementary base sequences may be complementary at each position in sequence by using standard base pairing (e.g., G:C, A:T or A:U pairing) or may contain one or more residues (including abasic residues) that are not complementary by using standard base pairing, but which allow the entire sequence to specifically hybridize with another base sequence in appropriate hybridization conditions. Contiguous bases of an oligomer are preferably at least about 80% (81, 82, 83, 84, 85, 86, 87, 88, 89, 90, 91, 92, 93, 94, 95, 96, 97, 98, 99, 100%), more preferably at least about 90% complementary to the sequence to which the oligomer specifically hybridizes. Appropriate hybridization conditions are well known to those skilled in the art, can be predicted readily based on sequence composition and conditions, or can be determined empirically by using routine testing (see Sambrook et al., Molecular Cloning, A Laboratory Manual, 2^{nd} ed. (Cold Spring Harbor Laboratory Press, Cold Spring Harbor, N.Y., 1989) at §§ 1.90-1.91, 7,37-7.57, 9.47-9.51 and 11.47 11.57, particularly at §§ 9.50-9.51, 11.12-11.13, 11.45-11.47 and 11.56-11.57).

As used herein, a “primer” defines an oligonucleotide which is capable of annealing to a target sequence, thereby creating a double-stranded region which can serve as an initiation point for nucleic acid synthesis under suitable conditions. Primers can be, for example, designed to be specific for certain alleles so as to be used in an allele-specific amplification system

A “probe” is meant to include a nucleic acid oligomer that hybridizes specifically to a target sequence in a nucleic acid or its complement, under conditions that promote hybridization, thereby allowing detection of the target sequence or its amplified nucleic acid, Detection may either be direct (i.e., resulting from a probe hybridizing directly to the target or amplified sequence) or indirect (i.e., resulting from a probe hybridizing to an intermediate molecular structure that links the probe to the target or amplified sequence). A probe's “target” generally refers to a sequence within an amplified nucleic acid sequence (i.e., a subset of the amplified sequence) that hybridizes specifically to at least a portion of the probe sequence by standard hydrogen bonding or “base pairing.” Sequences that are “sufficiently complementary” allow stable hybridization of a probe sequence to a target sequence, even if the two sequences are not completely complementary. A probe may be labeled or unlabeled.

A “label” refers to a molecular moiety or compound that can be detected or can lead to a detectable signal. A label is joined, directly or indirectly, to a nucleic acid probe or the nucleic acid to be detected (e.g., an amplified sequence). Direct labeling can occur through bonds or interactions that link the label to the nucleic acid (e.g., covalent bonds or non-covalent interactions), whereas indirect labeling can occur through use a “linker” or bridging moiety, such as additional oligonucleotide(s), which is either directly or indirectly labeled. Bridging moieties may amplify a detectable signal. Labels can include any detectable moiety (e.g., a radionuclide, ligand such as biotin or avidin, enzyme or enzyme substrate, reactive group, chromophore such as a dye or colored particle, luminescent compound including a bioluminescent, phosphorescent or chemiluminescent compound, and fluorescent compound). Preferably, the label on a labeled probe is detectable in a homogeneous assay system, i.e., in a mixture, the bound label exhibits a detectable change compared to an unbound label.

Polymerase chain reaction (PCR). PCR is carried out in accordance with known techniques. See, e.g., U.S. Pat. Nos. 4,683,195; 4,683,202; 4,800,159; and 4,965,188 (the disclosures of all three U.S. Patents are incorporated herein by reference). In general, PCR involves a treatment of a nucleic acid sample (e.g., in the presence of a heat stable DNA polymerase) under hybridizing conditions, with one oligonucleotide primer for each strand of the specific sequence to be detected. An extension product of each primer which is synthesized is complementary to each of the two nucleic acid strands, with the primers sufficiently complementary to each strand of the specific sequence to hybridize therewith. The extension product synthesized from each primer can also serve as a template for further synthesis of extension products using the same primers. Following a sufficient number of rounds of synthesis of extension products, the sample is analyzed to assess whether the sequence or sequences to be detected are present. Detection of the amplified sequence may be carried out by visualization following like, for example, ethidium bromide (EtBr) staining of the DNA following gel electrophoresis, or using a detectable label in accordance with known techniques, and the like. For a review on PCR techniques (see PCR Protocols, A Guide to Methods and Amplifications, Michael et al. Eds, Acad. Press, 1990).

“Amplification” refers to any known in vitro procedure for obtaining multiple copies (“amplicons”) of a target nucleic acid sequence or its complement or fragments thereof. In vitro amplification refers to production of an amplified nucleic acid that may contain less than the complete target region sequence or its complement Known in vitro amplification methods include, e.g, transcription-mediated amplification, replicase-mediated amplification, polymerase chain reaction (PCR) amplification, ligase chain reaction (LCR) amplification, and strand-displacement amplification (SDA). Replicase-mediated amplification uses self-replicating RNA molecules, and a replicase such as Qβ-replicase (e.g., Kramer et al., U.S. Pat. No. 4,786,600). PCR amplification is well known and uses DNA polymerase, primers, and thermal cycling to synthesize multiple copies of the two complementary strands of DNA or cDNA (e.g., Mullis et al., U.S. Pat. Nos. 4,683,195, 4,683,202, and 4,800,159). LCR amplification uses at least four separate oligonucleotides to amplify a target and its complementary strand by using multiple cycles of hybridization, ligation, and denaturation (e.g., EP Pat. App. Pub. No, 0 320 308). SDA is a method in which a primer contains a recognition site for a restriction endonuclease that permits the endonuclease to nick one strand of a hemimodified DNA duplex that includes the target sequence, followed by amplification in a series of primer extension and strand displacement steps (e.g., Walker et al., U.S. Pat. No. 5,422,252). Another known strand-displacement amplification method does not require endonuclease nicking (Dattagupta et al., U.S. Pat. No. 6,087,133). Transcription-mediated amplification is used in the present invention. Those skilled in the art will understand that the oligonucleotide primer sequences of the present invention may be readily used in any in vitro amplification method based on primer extension by a polymerase (see generally Kwoh et al., 1990, Am. Biotechnol. Lab., 8:14-25; Kwoh et al., 1989, Proc. Natl. Acad. Sci. U.S.A., 86:1173-1177; Lizardi at al., 1988, BioTechnology 6:1197-1202; Malek et al., 1994, Meth. Mol. Biol., 28:253-260; and Sambrook et al., 2000, Molecular Cloning—A Laboratory Manual, Third Edition, CSH Laboratories). As commonly known in the art, the oligonucleotides are designed to bind to a complementary sequence under selected conditions.

An “immobilized probe” or “immobilized nucleic acid” refers to a nucleic acid that joins, directly or indirectly, a capture oligomer to a solid support. An immobilized probe is an oligomer joined to a solid support that facilitates separation of bound target sequence from unbound material in a sample. Any known solid support may be used, such as matrices and particles free in solution, made of any known material (e.g., nitrocellulose, nylon, glass, polyacrylate, mixed polymers, polystyrene, silane polypropylene and metal particles, preferably paramagnetic particles). Preferred supports are monodisperse paramagnetic spheres (i,e., uniform in size ± about 5%), thereby providing consistent results, to which an immobilized probe is stably joined directly (e.g., via a direct covalent linkage, chelation, or ionic interaction), or indirectly (e.g., via one or more linkers), permitting hybridization to another nucleic acid in solution.

Fluorometric detection. Upon excitation with light, certain molecules emit photons or excitons of lesser energy (different wavelength). Hence, several fluorescent molecules have found applications as reporters than can be detected and quantified, after excitation at a suitable wavelength, with several apparatuses such as fluorometers, confocal fluorescence scanners, microscopes, etc.

Colorimetric detection. This mode of detection refers to methods that produce liquid color changes or yield colored precipitates that can be monitored by e.g. spectrophotometry, flatbed scanning, microscopy, or by the naked eye.

Electrochemical detection. Generally performed at the surface of electrodes, oxydo-reduction reactions of reporter molecules yield electrons that can be monitored using suitable apparatus such as potentiostats.

Chemiluminescent detection. Chemiluminescence is a property exhibited by several reporter systems relying on enzymes such as alkaline phosphatase or horseradish peroxidase, which convert a substrate with concomitant emission of light that can be detected by autoradiography (solid phase) or luminometry (liquid phase).

Examples of “solid support surfaces” include without limitation glass, fiberglass, plastics such as polycarbonate, polystyrene or polyvinylchloride, complex carbohydrates such as agarose and Sepharose™, acrylic resins such as polyacrylamide and latex beads, metals such as gold. Other suitable solid supports include microtiter plates, magnetic particles or a nitrocellulose or other membranes. Techniques for coupling antibodies to such solid supports are well known in the art (Weir et al., “Handbook of Experimental Immunology” 5th Ed., Blackwell Scientific Publications, Oxford, England, (1996); Jacoby et al., Meth. Enzymol, 34 Academic Press, N.Y. (1974)).

As used herein, “chemical derivatives” is meant to cover additional structurally related chemical moieties not explicitly disclosed herein which may have different physico-chemical characteristics (e.g. solubility, absorption, half life, decrease of toxicity and the like).

The term “sample” should be should be construed herein to include without limitation a biological sample, or any other material or portion derived therefrom which may contain the target nucleic acid or protein.

The term “positively charged reporter” or “reporter” should be construed herein to include without limitation transition metal cations, cationic polymers with affinity for nucleic acids such as polythiophenes (monomer structure shown in FIG. 1A) and derivatives.

### BRIEF DESCRIPTION OF THE FIGURES

FIG. 1 shows a schematic description and experimental results of the fluorometric detection on microarrays using a cationic polythiophene transducer in the presence of a) single-stranded oligonucleotide; b) hybridized oligodeoxyribonucleotides; c) neutral PNA, and d) hybridized duplex PNA-oligonucleotide. Panel A describes the probe-target combinations that were tested for fluorometric detection using a cationic polythiophene transducer while Panel B shows the relative fluorescence signal intensity following reaction of the cationic polythiophene transducer in the presence of the DNA-DNA and PNA-DNA complexes generated by hybridization onto a microarray. Note the low fluorescence signal intensity following reaction of the PNA probes with the cationic polythiophene transducer (c) compared to the signal obtained in a similar experiment done against DNA probes (a), demonstrating the utility of PNAs for detection of unlabeled DNA molecules.

FIG. 2 shows specificity of oligodeoxyribonucleotide hybridization to PNA probes when polymeric detection is used as transducer. Hybridizations were performed at room temperature with a concentration of 7.5×10¹⁰ targets per μL. Hybridization of PNA probes to perfectly complementary, or complementary oligonucleotides presenting a terminal mismatch, a central mismatch, or two mismatches were performed in triplicate. Fluorescence intensities from hybridized probes were corrected by substraction of background fluorescence intensity.

### DESCRIPTION OF EMBODIMENTS OF THE INVENTION

U.S. provisional application No. 60/528,748, which is the priority document of the present application, is incorporated by reference herein.

In an embodiment, the present invention relates to methods for the detection of nucleic acids specifically hybridized to neutral nucleic acid analog oligomers such as probes. In an embodiment, these probes are immobilized onto a support.

The foregoing method comprises: exposing uncomplexed neutral probes to a sample possibly containing complementary nucleic acid targets; submitting this mixture to physicochemical conditions compatible with nucleic acids hybridization wherein single-stranded nucleic acids bind specifically to complementary neutral probe(s) by a hybridization process; submitting this negatively charged capture probe-nucleic acid target hybrids to a positively charged reporter, such as transition metal atoms, molecules, or macromolecules, capable of recognizing and electrostatically binding the ribose-phosphate backbone of the hybridized nucleic acid targets; and detecting higher-order complexes of reporters bound to the aforementioned hybrids using detection methods, non limiting examples of which are: optical, fluorescence, or electrochemical detection.

In an embodiment, the target nucleic acids are released from microbial and/or eucaryotic cells or from viral particles potentially present in the sample. The target nucleic acids may be generated by nucleic acid amplification procedures, non-limiting examples of which are: polymerase chain reaction (PCR), reverse transcriptase-PCR (RT-PCR), strand displacement amplification (SDA), as well as by chemical synthesis. The reporters exhibit low affinity for uncharged probes, thereby allowing to minimize non-specific background signal.

In an embodiment, the uncharged probes are made of PNA or of methylphosphonate; the nucleic acid targets are made of DNA or RNA molecules; and the nucleic acid targets are generated by PCR.

In another embodiment, the neutral probes are capture probes bound to a surface such as glass surfaces, electrode surfaces, particles surfaces, gel matrix, membrane surfaces, paper surfaces, and plastic surfaces.

In an embodiment, the present invention relates to a method using reporters (such as water-soluble cationic polymers for example) as transducers for the hybridization of unlabeled nucleic acids to neutral nucleic acid analog probes. Nucleic acids are used in the present invention as scaffolds for the generation of polythiophene polymer complexes.

The phosphate groups of hybridized DNA or RNA offer a high concentration of negatively-charged groups that can attract positively charged metallic ions (Rossetto et al., 1994 J. Inorganic Biochem., 54:167-186) from which detectable or quantifiable complexes can be elaborated, ideally in physical or chemical conditions that will have minimal effects on the stability of PNA-nucleic acid duplexes. Nucleic acids are used in the present invention as scaffolds for the in situ synthesis or self-assembly of metallic complexes.

Silver staining is a method that has been used to detect several types of macromolecules (DNA, RNA, proteins, etc.). DNA metallization is a process that relies on the affinity of silver ions (Ag⁺) for negatively charged nucleic acids before a reduction step that yields metallic silver (Ag⁰), detectable by microscopy or colorimetric methods, or electrical means. In the process described by Braun et al. (1998), silver ions were used to construct a nanowire between two electrodes joined by adenovirus DNA, hybridized by its extremities to both electrodes. The hybridized DNA was reacted with Ag₊ and reduced to Ag⁰ by an isothermal photographictype process using a hydroquinone, upon demonstration of the usefulness of PNA for detection of hybridized nucleic acids on biochips, a colorimetric detection approach, relying on microscopy or digital scanning, is favored (Braun et al., 1998, Nature, 391:775-778).

Cadmium ions (Cd⁺⁺) are also thought of as having affinity for nucleic acids. Cd⁺² is also an important ion for the synthesis of photoactive (fluorescent or luminescent) quantum dots following exposure of complexed Cd⁺⁺ to a source of sulfur ions (H₂S or Na₂S). In respect to ideal physical or chemical conditions for hybridized duplexes, cadmium sulfide particles are the only quantum dots that were shown to be safely assembled on nucleic acids or anionic polymers (Coffer et al., 1996, Appl. Phys. Lett., 69:3851-3853; Huang et al., 1996, Polym. Bull., 36:337-340; Storhoff and Mirkin, 1999, Chem. Rev., 99:1849-1862). For the detection of nucleic acids however, microscopy methods will be more useful than spectrophotometric methods since low-temperature synthesis is prone to generate particles of non homogeneous sizes, the emission spectra of CdS quantum dots being highly dependent on the size of the nanoparticles.

Several enzymes are known to recognize and chemically or physically modify the structure of nucleic acids. Alkaline phosphatase is a DNA-modifying enzyme that is used to dephosphorylate the extremities of nucleic acid molecules. In preliminary experiments, it was observed that alkaline phosphatase and polystyrene beads conjugated to alkaline phosphatase have affinity for DNA molecules. Further, alkaline phosphatase permits the detection by colorimetric, fluorescent, and chemiluminescent methods which are either economical or extremely sensitive by allowing signal amplification.

The use of systems for the detection of hybridized nucleic acids comprises the following steps: exposing uncomplexed neutral probes to a sample mixture possibly containing complementary nucleic acid targets; submitting this mixture to conditions favorable to hybridization of the probes to the nucleic acids contained in the sample; submitting a reporter atom, molecule or macromolecule (e.g. water-soluble cationic polythiophene; enzyme serving as transducer) to the hybridized microarray; and detection of higher order complexes (e.g. fluorometric, colorimetric, electrochemical) using an appropriate apparatus (e.g. confocal fluorescence scanner, epifluorescence microscope, potentiostat, etc.) or direct observation (e.g. naked eye).

The before mentioned probes can be capture probes immobilized onto a surface that can be chemically modified glass, silicon, gold, as well as other surfaces as will be easily understood by the person having ordinary skill in the art. The surface can be planar, spherical, or provided in any suitable configuration as is known in the art The surface can also be an electrode. Glass, silicon, or plastic surfaces can be functionalized with various chemicals to yield aldehyde, amino, epoxy, or carboxyl moieties that can be activated with carbonyldiimidazole compounds or another suitable compound, making them capable of reacting with oligonucleotides bearing terminal amino groups, as is known in the art. The uncomplexed neutral capture probes can be PNA, methylphosphonate, as well as other neutral capture probes known to the skilled artisan. These uncomplexed neutral capture probes can also be immobilized onto the surface. Neutral capture probe can be synthesized to contain terminal amino, thiol, carboxyl, or any other suitable functional group that is used to create chemical bonds to surfaces. The surface can be coated or passivated with different agents, such as polyethylene glycol or BSA, to prevent non-specific binding of the analyte nucleic acids. The sample can be nucleic acids extracted from microbial or eucaryotic cells or from viral particles. A wide variety of methods for cell lysis and nucleic acid isolation from microbes have been extensively described in the literature (e.g. Nolte and Caliendo, 2003, Molecular detection and identification of microorganisms, pp. 234-256, In Manual of Clinical Microbiology (8^{th} ed.), Murray et al., American Society for Microbiology, Washington, D.C.; Jungkind and Kessler, 2002, Molecular methods for diagnosis of infectious diseases, pp. 306-323, In Manual of Commercial Methods In Clinical Microbiology, Truant, American Society for Microbiology, Washington, D.C.). Protocols for nucleic acid preparation from a variety of microbial cells are disclosed in WO 03/008636. Furthermore, there are many commercially available kits for nucleic acid extraction from various types of cells including microbial cells. WO 03/008636 discloses a comparison of popular commercial kits for rapid nucleic acid extraction from different microbial cultures. The target unlabeled anionic nucleic acid may be generated by molecular amplification techniques. The molecular amplification technique can be PCR, RT-PCR, as well as other amplification techniques known in the art (Nolte and Caliendo, 2003, Molecular detection and identification of microorganisms, p. 234-256, In Manual of Clinical Microbiology (8^{th} ed.), Murray et al., American Society for Microbiology, Washington, D.C.; Frecdicks and Relman, 1999, Clin. Infect Dis., 29:475-488).

The before mentioned favorable conditions for hybridization can be performed, in accordance with an embodiment of the invention, using various time scales, temperatures, as well as various hybridization devices (e.g. hybridization chambers, microfluidic systems, immersion in a liquid, etc.). In an embodiment, the conditions may involve shaking of the mixture. In another embodiment, there is no shaking of the mixture. The conditions may include the use of electric or magnetic fields, The conditions can include different compositions of hybridization solutions. The hybridization solution can be buffers or salt solutions of various concentrations and composition (e.g. salt sodium citrate, salt sodium phosphate EDTA, sodium phosphate, sodium acetate, etc.), as well as solutions that may contain anionic, cationic, zwitterionic or uncharged detergents (e.g. SDS, Igepal CA630, Triton, Tween-20, etc.). The hybridization solutions may also contain chaotropic agents. (e.g. formamide, urea, guanidine, etc.), various additives that can modify hybridization behavior (e.g. betaine, TMAC, etc.), blocking and background reducing agents (e.g. BSA, PVP, etc.), and/or various additives that have a positive impact on specificity, sensitivity, and speed of hybridization. The hybridization solution can also be water. The hybridized microarray may or may not be washed following hybridization. The washing can be done in conditions as diverse as for the hybridization reaction conditions.

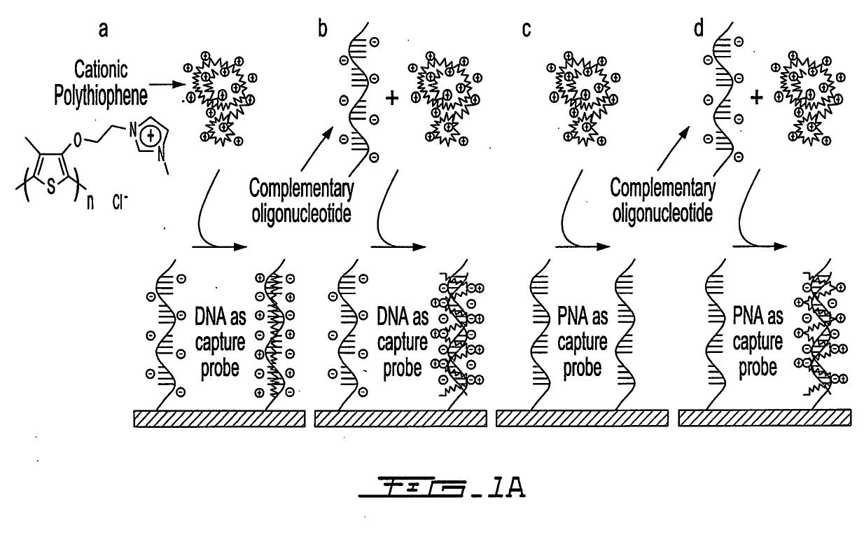

The before mentioned reaction of the reporter can be carried out in various conditions such as for the hybridization reaction. In an embodiment, the reporter comprises a water-soluble cationic polythiophene (see FIG. 1A). The reporter electrostatically binds to the hybridized negatively-charged target while it has no significant interaction with the capture probes. This reaction is followed by appropriate washes. The washes can be done under various conditions as described for the hybridization reaction.

In an embodiment, the before mentioned detection of higher order complexes comprises fluorometric detection.

During detection, the absence of a signal implies non-hybridization and as such the absence of the target nucleic acid in question. Contrarily, a signal implies hybridization and as such the presence of the targeted nucleic acid within the sample.

In another embodiment, the uncomplexed neutral probes can be exposed to a sample mixture possibly containing complementary nucleic acid targets and a reporter atom, molecule or macromolecule (e.g. water-soluble cationic polythiophene, enzymes) serving as a transducer. The probes can be capture probes immobilized onto a surface. In an embodiment, the reporter is a water-soluble cationic polythiophene. The reporter electrostatically binds to the hybridized negatively-charged target while it has no significant interaction with the capture probes.

Detection (for example and without limitation: fluorometric, colorimetric, electrchemical) is conducted using an appropriate apparatus (e.g. confocal fluorescence scanner, epifluorescence microscope, potentiostat, etc.).

The present label-free detection methodology can be applied to existing microarray technologies.

A non-limiting embodiment of the invention is illustrated in Example 1 using cationic, water-soluble conjugated polymers with neutral PNA capture probes attached to glass surface. This resulted in a larger affinity contrast between non-hybridized PNA probes (neutral state) and hybridized PNA-DNA spots (the substrates becoming negatively-charged).

Improvements in terms of sensitivity and overall performance can be obtained by exciting and detecting the polymeric fluorescent transducer at the optimal wavelength, reducing the size of the spots, the volume for hybridization reactions, and by detecting larger DNA molecules (e.g. PCR amplicons) since the amount of complexed polymeric fluorescent transducer will be increased through electrostatic interactions. This remarkably simple methodology opens exciting possibilities for biomedical research and DNA diagnostics. Also, the electroactivity in aqueous solutions of the present polythiophene derivative can be exploited for the electrical detection of nucleic acid hybridization events

The invention Will be further described by way of the following examples, which serve to illustrate the invention only and by no means limit its scope.

### EXAMPLES

### Example 1

### Detection of Target Oligonucleotide DNA Using Fluorescent Cationic Polymers and PNA Capture Probes

One of the possible avenues for molecular diagnostics is the use of microarrays to screen for the presence of specific nucleic acid sequences. One of the key criteria for a good diagnostic kit is speed and one of the steps limiting the speed of microarray hybridization is the necessity of target nucleic acids labeling and amplification. To alleviate those steps, two breakthroughs are necessary: a sensitive enough technology that allows near-single-molecule detection of nucleic acids and a method to detect unlabeled target nucleic acids. Novel cationic, water-soluble polythiophene derivatives can transduce DNA hybridization into a detectable signal (e.g. optical, fluorescent or electrochemical signal) (Pending patent application PCT/CA02/00485). Since such cationic polymer binds electrostatically to negatively-charged nucleic acids, neutral nucleic acid analogs such as PNA allow to reduce background signal.

Poly (1H-Imidazolium, 1-methyl-3-[2-[(4-methyl-3-thieny)oxy]ethyl]-chloride) was prepared as previously published (Ho et al., 2002, Angew. Chem. Int. Ed., 41:1548-1551). Oligodeoxyribonucleotide capture probes having a 5′ amino-linker modification were synthesized by Biosearch Technologies (Novato, Calif.). The amino-linker modification permits the covalent attachment of probes onto functionalized glass surface. PNA probes having a 5′ amine and two O linkers were synthesized by Applied Biosystems (Foster City, Calif.), The capture DNA or PNA probe of 15-mer (5′-CCGCTCGCCAGCTCC-3′) targeted a polymorphic region of the bla_{SHV-1} gene associated with β-lactam antibiotics resistance. Target oligonucleotides (i) fully complementary to the capture DNA or PNA probe (5′-GGAGCTGGCGAGCGG-3′), (ii) having two mismatched bases (5′-GGCGCTGACGAGCGG3′) and (iii) having a central single mismatch (5′-GGAGCTGACGAGCGG-3′) synthesizes by Biosearch Technologies were used.

Preparation of glass slides. All chemical reactions were carried out in polypropylene jars. Surfaces used were 25 mm×75 mm glass micro slides (VWR Scientific, West Chester, Pa.). After sonication (1 hour) in deionized water, the slides were again sonicated in 40 mL of 10% sodium hydroxyde (NaOH) for 1 hr, washed several times with deionized water, and dried under a stream of nitrogen. The slides were then sonicated in an aminopropyltrimethoxysilane solution (2 mL water, 38 mL; methanol and 2 mL aminopropyltrimethoxysilane) for 1 hr, washed with methanol, dried, and baked at 110° C. for 15 min. The amine modified slides were activated by sonication in 40 mL of 1,4-dioxane containing 0.32 g (2 mmol) of carbonyldiimidazole as coupling agent, washed with dioxane and diethyl ether, and dried under a stream of nitrogen.

Microarray production. The probes were diluted two-fold by the addition of Array-it Microspoting Solution Plus (Telechem International, Sunnyvale, Calif.), to a final concentration of 5 μM. Probes were spotted in triplicate, using a SDDC-2 arrayer (formerly VIRTEK, now Bio-Rad Laboratories, Hercules, Calif.) with SMP3 pins (TeleChem International, Sunnyvale, Calif.). Upon spotting, each volume of 0.6 nL spanned a diameter of 140-150 μm and contained about 1.8×10⁹ amino-modified probes. After spotting, slides were dried overnight, washed by immersion in boiling 0.1% SDS for 5 min, rinsed in ultra-pure water for 2 min, and dried by centrifugation for 5 min under vacuum (SpeedVac plus; Thermo Savant, Milford, Mass.). Slides were stored at room temperature in a dry, oxygen-free environment

DNA microarray hybridization, polymeric detection and data acquisition. Prehybridization and hybridization were performed in 15×13 mm Hybri-well self-sticking hybridization chambers (Sigma-Aldrich; St, Louis, Mo.). Microarrays were first prehybridized for 30 min at room temperature in 20 μL of 1× hybridization solution (6×SSPE [Omnipur, EM Science, Gibstown, N.J.], 0.03% polyvinylpyrrolidone [PVP], and 30% formamide). Subsequently, the prehybridization buffer was blown out of the chambers and replaced with the same buffer containing the target oligonucleotide at a final concentration of 2.5 μM. Hybridization was carried out at 22° C. for 15 min. After hybridization, the liquid was expelled from the chambers and replaced by a polymer solution. After a 15 min incubation period, the slides were washed with deionized water containing 0.1% Igepal CA630 (Sigma-Aldrich, St. Louis, Mo.). Then, microarrays were dried by centrifugation at 3000 rpm for 3 minutes. Slides were scanned using the Cy3 configuration of ScanArray 4000XL (formerly GSI Lumonics, now Packard Bioscience Biochip Technologies, Billerica, Mass.) and the fluorescent signals were analyzed using QuantArray software (formerly GSI Lumonics, now Packard Bioscience Biochip Technologies).

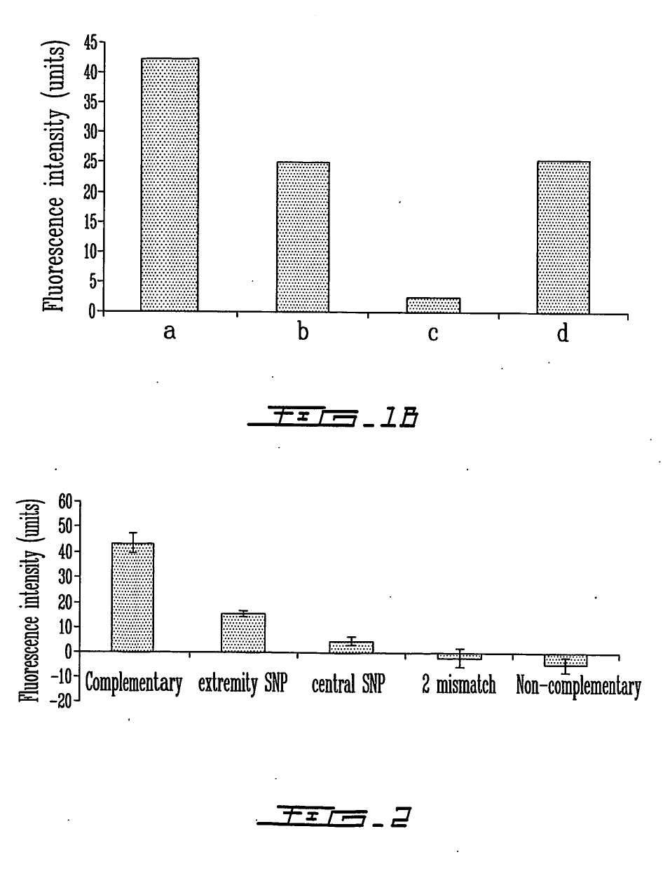

Traditional DNA microarrays are relatively straightforward to design and build, but conditions for spotting and grafting PNA probes to glass or silica surfaces are less documented. Also, experiments carried out with commercially available aldehyde-functionalized glass slides (CEL Associates, Pearland, Tex.) permitted Cy3-labeled oligonucleotides detection, but gave no or poor signal when detection was conducted using our polymeric biosensor (i.e. a polythiophene derivative). To resolve this challenge, central to the utilization of polythiophene transducers on PNA microarrays, glass derivatization was explored. Two promising glass functionalization methods were developed and permitted the comparison between commercial aldehyde slides, aminoalkylsilane slides activated with carbonyldiimidazole (FIG. 2) and “dendrimeric” slides (Beaucage, 2001, Curr. Med. Chem., 8:1213-1244; Beier et al., 1999, Nucleic Acids Res. 27:1970-1977). Cy3-labeled targets and polymeric detection were both tested on each type of functionalized slide. A significant increase in hybridization signal when aminoalkylsilane slides were used for labeled targets detection experiments was observed. Also, those slides allowed the use of the polymeric biosensor, which was not allowed by aldehyde slides and poorly supported by dendrimeric slides (data not shown). Aminated slides activated by carbodiimidazole were used to immobilize DNA and PNA capture probes for all experiments described, hereafter.

In the case of ssDNA capture probes (FIG. 1a) or target/probe dsDNA duplexes (FIG. 1b), the spots became fluorescent due to the formation of DNA-polythiophene complexes. However, discrimination between hybridized and non-hybridized spots was difficult using a conventional microarray detection apparatus. On the contrary, the cationic polymer did not bind to neutral PNA capture probes (FIG. 1c). In the case of target oligonucleotide DNA probes hybridized to capture PNA probes, the polymer binds to negatively-charged DNA and allows the transduction of hybridization into fluorescence (FIG. 1D). These results are consistent with those previously reported by Gaylord et al. (Gaylord et al., 2002, Proc. Natl. Acad. Sci. U.S.A., 99:10954-10957) using cationic polyfluorene derivatives and PNA probes in aqueous solutions. These results clearly demonstrate the appropriateness of PNA capture probes for the detection of hybridization events with a positively-charged fluorescent polythiophene.

Specificity of detection was investigated by hybridizing mismatched oligonucleotides to PNA probes. After room temperature hybridization of oligonucleotides with PNA probes, the fluorescent polythiophene gave a strong signal over background when target oligonucleotide was fully complementary to the capture probe. Double-mismatched oligonucleotides and non complementary oligonucleotides produced near-background signals easily distinguishable from the much stronger signal observed with perfectly matched hybrids (21 X) (FIG. 2). For single mismatch, discrimination is strongly related to the position of the mismatch in the probe. When mismatch is located at the probe extremity, the signal intensity is reduced 2.5 fold compare to the perfect match. By contrast, a ratio of 6 is observed when the mismatch is located close to the center of the probe (FIG. 2). For hybridization in liquid phase, the differential excitation of complementary and mismatched dsDNA/polythiophene triplexes have been used to discriminate single nucleotide polymorphisms (SNP) (Ho et. al., 2002, Angew. Chem. Int. Ed., 41:1548-1551; Nilsson et al., 2003, Nat. Mater,. 2:419-424) However, for hybridization onto solid support, single nucleotide polymorphism (SNP) discrimination relies on the specificity of the PNA capture probes. The discrimination of oligonucleotides having two mismatches was possible using the standard procedure (FIG. 2). Also, the current method allowed the discrimination of SNP upon a wash at 55° C. (FIG. 2).

The analytical sensitivity of the detection scheme described here is approximately 1.5×10¹¹ molecules in a volume of 20 μL (2.5×10⁻¹³ moles or 7.5×10⁹ molecules/μL). In a recent report, Nilsson and Inganäs (Nat Mater., 2003, 2:419-424), have described the use of a zwitterionic polythiophene derivative able to detect 2×10⁻⁸ mole of oligonucleotide within a hydrogel matrix. This approach, based on standard glass microarray technologies, is presently five orders of magnitude more sensitive. Moreover, further progress in terms of sensitivity is obtained by reducing the size of the spots and the hybridization reaction volumes. Also, the detection of larger DNA molecules (e.g. amplicons) increases sensitivity since the amount of complexed fluorescent polymer is theoretically proportional to the amount of possible electrostatic interactions. Indeed, recent optimizations of the fluorometric detection applied to the polymer described herein has enabled the detection of only few hundred molecules of genetic materials in aqueous solutions. This clearly indicates that cationic conjugated polymers are highly sensitive fluorescent transducers (Doré et al., 2004, J. Am. Chem. Soc., 126:4240-4244). It is worth notice that the complex between polythiophene and PNA/DNA duplex was detected despite not being excited at the maximum absorption wavelength of the polythiophene (430 nm). Excitation at 550 nm using a standard slide scanner (e.g. ScanArray 4000XL from Packard Bioscience Biochip Technologies) was used for all experiments using the polythiophene biosensor fluorescence detection described in the present invention. Therefore, detection using this polymeric biosensor was far from optimal because of the unavailability of an appropriate laser for excitation (i.e. around 430 nm). It is estimated that the fluorescence signal measured at 550 nm is less than 5% of the fluorescence signal that would be detected using a 430 nm laser. Clearly, a more suitable excitation source greatly improves the analytical sensitivity of the polythiophene biosensor. A scanner modified to accommodate a non standard 430 nm laser is being fabricated by collaborators. The development of scanners specifically fabricated for detection using the polythiophene derivatives of the invention contribute to increase the analytical sensitivity.

In a recent study, Gaylord et al., have shown detection in solution of a complementary DNA hybridized to a PNA probe using Förster resonance energy transfert (FRET) between a water soluble conjugated polymer and a PNA probe labeled with a reporter chromophore (Gaylord et al., 2002, Proc. Natl. Acad. Sci. U.S.A., 99:10954-10957). In this work, similar results are shown without the need for labeled PNA. Moreover, it is demonstrated that this detection can be performed on PNA probes tethered onto a solid support. Those results show that this electrostatic strategy can also be used with other DNA detection methods such as electrochemistry (Liu and Anzai, 2004. Anal. Chem., 76:2975-2980), silver staining (Braun et al., 1998, Nature, 391:775-778; Brust and Kiely, 2002, Colloids Surfaces, 202:175-186), metallization (Warner and Hutchison, 2003, Nat. Mater., 2:272-277; Storhoff and Mirkin, 1999, Chem. Rev., 99:1849-1862), quantum dots (Alivisatos, 2000, Pure Appl. Chem., 72:3-9; Chan and Nie, 1998, Science, 281:2016-2018; Penner, 2000, Acc. Chem. Res., 33:78-86), or electrochemical dyes (Kricka, 2002, Ann. Clin. Biochem., 39:114-129). In conclusion, this approach to DNA detection on solid support is simple, specific and does not require labeling of the analyte prior to hybridization. This remarkably simple methodology is useful for genetic analysis applied for the diagnosis of infections, identification of genetic mutations, and forensic inquiries. For instance, this technology would be useful for the identification of pathogens and related antimicrobial resistance genotypes using microarrays. Finally, the electroactivity of the present polythiophene derivative is useful for a real-time electrical discrimination of SNPs on solid support.

### Example 2

### Detection of Target PCR Amplicon DNA Using Fluorescent Cationic Polymers and PNA Capture Probes.

Same as example 1, except that hybridization to the capture PNA or DNA probes were performed using 160 base pairs (bp) amplicons produced by asymmetric PCR. Recently, Germini et al. also reported that the hybridization of amplicons to PNA probes was more efficient with single-stranded PCR products (Germini et al., 2004, J. Agric. Food Chem., 52:4535-4540). Hybridization was performed exactly as described above for oligonucleotides except that the hybridization time was extended to one hour at 22° C. The amplicon at the final concentration of 2.9 nM in standard hybridization buffer (described above) was used for the hybridization. As shown in Example 1 for detection of a complementary DNA oligonucleotide, detection of single-stranded amplicon with the polymer biosensor was demonstrated when hybridized to a PNA probe (data not shown).

PCR amplifications were performed from 1 μl of a bacterial genomic DNA preparation at 1 ng/μl which was transferred directly to a 24-μl PCR mixture containing 50 mM KCl, 10 mM Tris-HCl (pH 9.0), 0.1% Triton X-100, 2.5 mM MgCl₂, 0.05 mM dNTP and 0.66 U of Taq DNA polymerase (Promega, Madison, Wis.). SHV-1 beta-lactamase gene was used as template. For the detection of amplicons, the following primers were used to synthesize 3 targets having different length and positioning on the probes. A centered target analyte was amplified using 0.4 μM of primer A (5′-CAGCTGCTGCAGTGGATGGT-3′) and 0.0114 μM of primer B (5′-GTATCCCGCAGATAAATCACCAC-3′). A target analyte with 3′ overhanging end oriented toward the solid support was amplified using 0.4 μM of primer A and 0.0114 μM of primer C (5′-CCGCTCGCCAGCTCC-3′) A target analyte with 5′ overhanging end oriented toward the liquid (buffer phase) was amplified using 0.4 μM of primers D (5′-GGAGCTGGCGAGCGG-3′) and 0.04 μM of B. PCR were performed using a PTC200 thermal cycler (MJ Research, Las Vegas, Nev.) using the following thermocycling conditions: denaturation at 94° C. for 180 sec 95° C., followed by 40 cycles of 96° C. for 1 sec; 60° C. for 30 sec. Finally, an extension step at 72° C. for 120 see was performed.

Hybridization were performed without prehybridization. The target DNA was denatured at 95° C. for 5 minutes and then chilled on ice for two minutes before being incorporated to the hybridization solution and introduced into the hybridization chamber (final concentration 2.9 nM). 16 hours or 1 hour hybridization were performed in the same conditions as for the target oligonucleotide hybridization. Washing, drying, and slide scanning were also performed as done for the oligonucleotide target.

The centered and solid support oriented amplicon gave a stronger signal (2.4 times above the background) than the liquid oriented amplicon (2 times above the background). This is predicted by our design rules (U.S. patent application 60/592,392).

Although the present invention has been described hereinabove by way of preferred embodiments thereof, it can be modified without departing from the spirit and nature of the subject invention as defined in the appended claims.

What is claimed is:

1. A method for detecting the presence of nucleic acids in a sample, said method comprising:
(a) exposing uncomplexed neutral capture probes to a sample possibly containing complementary nucleic acid targets, thereby generating a mixture;
(b) submitting said mixture to hybridization conditions which provide for said nucleic acids targets to bind specifically to complementary neutral capture probes, thereby generating negatively charged capture probe-nucleic acid target hybrids;
(c) submitting said negatively charged hybrids to positively charged reporters selected from group consisting of transition metal atoms, molecules, and macromolecules being capable of electrostatically binding to said hybrids, thereby generating higher-order complexes; and
(d) detecting said higher-order complexes.

2. A method according to claim 1, wherein said nucleic acids targets are unlabeled.

3. A method according to claim 1, wherein said capture probes are immobilized on a support surface.

4. A method according to claim 3; wherein said support surface is selected from the group consisting of a glass surface, a silicon surface, a gold surface, an electrode surface, a particle surface, a gel matrix, a membrane surface, a paper surface and a plastic surface.

5. A method according to claim 3, wherein said support surface comprises a solid support surface.

6. A method according to claim 5, wherein said solid support surface comprises a probe array.

7. A method according to claim 3, wherein said neutral capture probes are chemically modified to incorporate a functional group providing for said probes to covalently link to said surface.

8. A method according to claim 7, wherein said functional group is selected from the group consisting of amine, aldehyde, thiol, epoxy and carboxyl moieties.

9. A method according to claim 3, wherein said support surface is coated with a passivation agent preventing non-specific binding of nucleic acid targets.

10. A method according to claim 9, wherein said passivation agent is selected from the group consisting of polyvinylpyrollidone, polyethylene glycol, and BSA

11. A method according to claim 3, wherein said support surface is chemically modified, to facilitate coupling and chemical bonding of said neutral probe to said support surface.

12. A method according to claim 11, wherein said support surface is chemically modified to yield functional groups selected from the group consisting of an aldehyde, an aminoalkylsilane activated with carbonyldiimidazole, thiol, epoxy and carboxyl moieties.

13. A method according to claim 1, wherein said neutral capture probes are selected from the group consisting of peptide nucleic acids (PNA) and methylphosphonate.

14. A method according to claim 1, wherein said nucleic acid targets comprise DNA or RNA molecules.

15. A method according to claim 1, wherein said nucleic acid targets are generated by methods selected from the group consisting of polymerase chain reaction (PCR), reverse transcriptase-PCR (RT-PCR), strand displacement amplification (SDA), ligase chain reaction (LCR), transcription-associated amplification, nucleic acid sequence-based amplification (NASBA), whole genome amplification (WGA), helicase-dependent isothermal amplification, and chemical synthesis.

16. A method according to claim 1, further comprising a washing step after step (c).

17. A method according to claim 1, wherein said reporters serve as transducers.

18. A method according to claim 1, wherein said reporters exhibit low affinity for uncharged probes.

19. A method according to claim 1, wherein said reporters are capable of electrostatically binding to the phosphate backbone of said hybrids.

20. A method according to claim 1, wherein said transition metal atoms are selected from the group consisting of Ag⁺ and Cd⁺⁺.

21. A method according to claim 1, wherein said transition metal atoms comprise ions that can be chemically modified to yield higher-order complexes using bound nucleic acids as a scaffold.

22. A method according to claim 1, wherein said detection includes a chemical reaction step rendering said transition metal atoms detectable.

23. A method according to claim 1, wherein said reporters comprise polythiophenes.

24. A method according to claim 23, wherein said polythiophenes are water soluble and cationic.

24. A method according to claim 1, wherein said reporters comprise enzymes.

25. A method according to claim 24, wherein said enzymes comprise alkaline phosphatase having polystyrene beads conjugated thereto.

26. A method according to claim 1, wherein said detection is selected from the group consisting of optical detection, fluorometric detection, colorimetric detection, electrochemical detection, chemiluminescent detection, microscopy and spectrophotometric detection.

27. A method for detecting the presence of nucleic acids in a sample, said method comprising:
(a) exposing uncomplexed neutral capture probes to a sample possibly containing complementary nucleic acid targets and containing positively charged reporters selected from group consisting of transition metal atoms, molecules and macromolecules, thereby generating a mixture;
(b) submitting said mixture to hybridization conditions which provide for said nucleic acids targets to bind specifically to complementary neutral capture probes, thereby generating negatively charged capture probe-nucleic acid target hybrids, said reporters being capable of electrostatically binding to said hybrids, thereby generating higher-order complexes; and
(c) detecting said higher-order complexes.

28. A method according to claim 27, wherein said nucleic acids targets are unlabeled.

29. A method according to claim 1, wherein said capture probes are immobilized on a support surface.

30. A method according to claim 29, wherein said support surface is selected from the group consisting of a glass surface, a silicon surface, a gold surface, an electrode surface, a particle surface, a gel matrix, a membrane surface, a paper surface and a plastic surface.

31. A method according to claim 29, wherein said support surface comprises a solid support surface.

32. A method according to claim 31, wherein said solid support surface comprises a probe array.

33. A method according to claim 29, wherein said neutral capture probes are chemically modified to incorporate a functional group providing for said probes to covalently link to said support surface.

34. A method according to claim 33, wherein said functional group is selected from the group consisting of amine, aldehyde, thiol, epoxy and carboxyl moieties.

35. A method according to claim 29, wherein said support surface is coated with a passivation agent preventing non-specific binding of nucleic acid targets.

36. A method according to claim 35, wherein said passivation agent is selected from the group consisting of polyvinylpyrollidone, polyethylene glycol, and BSA.

37. A method according to claim 29, wherein said support surface is chemically modified, to facilitate coupling and chemical bonding of said neutral probe to said support surface.

38. A method according to claim 37, wherein said support surface is chemically modified to contain functional groups selected from the group consisting of an aldehyde, an aminoalkylsilane activated with carbonyldiimidazole, thiol, epoxy and carboxyl moieties.

39. A method according to claim 27, wherein said neutral capture probes are selected from the group consisting of peptide nucleic acids (PNA), and methylphosphonate.

40. A method according to claim 27, wherein said nucleic acid targets are selected from the group consisting of DNA and RNA molecules.

41. A method according to claim 27, wherein said nucleic acid targets are generated by methods selected from the group consisting of polymerase chain reaction (PCR), reverse transcriptase-PCR (RT-PCR), strand displacement amplification (SDA), ligase chain reaction (LCR), transcription-associated amplification, nucleic acid sequence-based amplification (NASBA), whole genome amplification (WGA), helicase-dependent isothermal amplification, and chemical synthesis.

42. A method according to claim 27, further comprising a washing step after step (b).

43. A method according to claim 27, wherein said reporters exhibit low affinity for uncharged probes.

44. A method according to claim 27, wherein said reporters are capable of electrostatically binding to the phosphate backbone of said hybrids.

45. A method according to claim 27, wherein said transition metal atoms are selected from the group consisting of Ag⁺ and Cd⁺⁺.

46. A method according to claim 27, wherein said transition metal atoms comprise ions that can be chemically modified to yield higher-order complexes using bound nucleic acids as a scaffold.

47. A method according to claim 27, wherein said detection includes a chemical reaction step rendering said transition metal cations detectable.

48. A method according to claim 27, wherein said reporters comprise polythiophenes.

49. A method according to claim 48, wherein said polythiophenes are water-soluble and cationic.

50. A method according to claim 27, wherein said reporters comprise enzymes.

51. A method according to claim 51, wherein said enzymes comprise alkaline phosphatase having polystyrene beads conjugated thereto.

52. A method according to claim 27, wherein said detection is selected from the group consisting of optical detection, fluorometric detection, colorimetric detection, electrochemical detection, chemiluminescent detection microscopy and spectrophotometric detection.

53. A kit for detecting the presence of nucleic acids in a sample, said kit comprising:
uncomplexed neutral capture probes;
a control sample possibly containing nucleic acid targets that are complementary to the neutral capture probes; and
one or more positively charged reporters selected from the group consisting of transition metal cations, molecules or macromolecules; said reporters being capable of electrostatically binding to negatively charged capture probe-nucleic acid target hybrids.

54. A kit according to claim 53, wherein said neutral capture probes are selected from the group consisting of peptide nucleic acids (PNA) and methylphosphonate.

55. A kit according to claim 53, wherein said capture probes are immobilized on a support surface.

56. A kit according to claim 55, wherein said support surface is selected from the group consisting of a glass surface, a silicon surface, a gold surface, an electrode surface, a particle surface, a gel matrix, a membrane surface, a paper surface and a plastic surface.

57. A kit according to claim 55, wherein said support surface comprises a solid support surface 58, A kit according to claim 57, wherein said solid support surface comprises a probe array.
